(19) United States
(12) Patent Application Publication
(10) Pub. No.: US 2024/0108313 A1
(43) Pub. Date: Apr. 4, 2024

(54) IMAGE PROCESSING DEVICE, IMAGE DISPLAY SYSTEM, IMAGE PROCESSING METHOD, AND IMAGE PROCESSING PROGRAM

(71) Applicant: TERUMO KABUSHIKI KAISHA, Tokyo (JP)

(72) Inventors: Yasukazu SAKAMOTO, Hiratsuka-shi (JP); Shunsuke YOSHIZAWA, Ebina-shi (JP); Clément JACQUET, Osaka (JP); Stephen TCHEN, Osaka (JP); Hector PITEAU, Osaka (JP); Edgar BAUCHER, Osaka (JP); Thomas HENN, Osaka (JP); Ryosuke SAGA, Osaka (JP)

(73) Assignee: TERUMO KABUSHIKI KAISHA, Tokyo (JP)

(21) Appl. No.: 18/476,513

(22) Filed: Sep. 28, 2023

(30) Foreign Application Priority Data
Sep. 30, 2022 (JP) 2022-157989

(51) Int. Cl. A61B 8/08; A61B 8/00

(52) U.S. Cl. CPC A61B 8/483; A61B 8/0858; A61B 8/466

(57) ABSTRACT

An image processing device includes: a control unit configured to acquire a cross-sectional image obtained using a sensor configured to move in a lumen of a biological tissue for each position in a movement direction of the sensor, analyze the acquired cross-sectional image to calculate a centroid position of a cross section of the lumen in the cross-sectional image, and execute smoothing on a calculation result obtained for a plurality of positions in the movement direction of the sensor, and evaluate a difference between the calculated centroid position and a centroid position obtained as a result of the smoothing for each position in the movement direction of the sensor, thereby deriving a pulsation score representing expansion and contraction of the lumen in the acquired cross-sectional image by a numerical value.

### CROSS-REFERENCES TO RELATED APPLICATIONS

This application claims priority to Japanese Patent Application No. 2022-157989 filed on Sep. 30, 2022, the entire content of which is incorporated herein by reference.

### TECHNOLOGICAL FIELD

The present disclosure generally relates to an image processing device, an image display system, an image processing method, and an image processing program.

### BACKGROUND DISCUSSION

U.S. Patent Application Publication No. 2010/0215238, U.S. Pat. Nos. 6,385,332, and 6,251,072 disclose a technique of generating a three-dimensional image of a cardiac cavity or a blood vessel by using an ultrasound (US) image system.

Image diagnosis that uses intravascular ultrasound (IVUS) is widely performed for an intracardiac cavity, a cardiac blood vessel, and a lower extremity arterial region, and the like. The IVUS is a device or a method that provides a two-dimensional image of a plane perpendicular to a longitudinal axis of a catheter.

Currently, it is necessary for a surgeon to perform an operation by imagining a three-dimensional structure in the head of the surgeon while sequentially observing two-dimensional images based on the IVUS, which is a barrier especially for young or inexperienced physicians. In order to remove such a barrier, it is conceivable to automatically generate a three-dimensional image showing a structure of a biological tissue such as the cardiac cavity or the blood vessel from the two-dimensional image based on the IVUS, and display the generated three-dimensional image to the surgeon.

A size and a shape of the biological tissue such as the cardiac cavity or the blood vessel temporally changes due to an influence of the pulsation of a heart. Since the two-dimensional images based on the IVUS used for generating the three-dimensional image are not all acquired at the same timings in a pulsation cycle, as a result, a three-dimensional image having unevenness may be drawn. It is difficult for the surgeon to accurately grasp the structure of the biological tissue based on such a three-dimensional image.

### SUMMARY

The present disclosure can help reduce an influence of a pulsation when generating a three-dimensional image representing a biological tissue, or provide information indicating a timing in a pulsation cycle at which a two-dimensional image that can be used for generating such a three-dimensional image is acquired.

Some aspects of the present disclosure are shown below.

[1] An image processing device includes: a control unit configured to acquire a cross-sectional image obtained using a sensor configured to move in a lumen of a biological tissue for each position in a movement direction of the sensor, analyze the acquired cross-sectional image to calculate a centroid position of a cross section of the lumen in the cross-sectional image, and execute smoothing on a calculation result obtained for a plurality of positions in the movement direction of the sensor, and evaluate a difference between the calculated centroid position and a centroid position obtained as a result of the smoothing for each position in the movement direction of the sensor, thereby deriving a pulsation score representing expansion and contraction of the lumen in the acquired cross-sectional image by a numerical value.

[2] The image processing device according to [1], in which the control unit determines whether to select an acquired cross-sectional image according to a derived pulsation score for each position in the movement direction of the sensor, and generates a three-dimensional image representing the biological tissue based on a selected image group obtained for at least some positions among the plurality of positions.

[3] The image processing device according to [2], in which for each position in the movement direction of the sensor, the control unit selects an acquired cross-sectional image, and uses the obtained selected image for generating or updating the three-dimensional image when a derived pulsation score is within a setting range, and does not select the cross-sectional image when the pulsation score is outside the setting range.

[4] The image processing device according to [2], in which for each position in the movement direction of the sensor, the control unit selects an acquired cross-sectional image, and uses the obtained selected image for generating or updating the three-dimensional image when a derived pulsation score is within a setting range, and processes the cross-sectional image and uses the obtained processed image for generating or updating the three-dimensional image when the pulsation score is outside the setting range.

[5] The image processing device according to [4], in which when an operation of changing the setting range is received, for each position in the movement direction of the sensor, the control unit uses an acquired cross-sectional image for updating the three-dimensional image when a derived pulsation score is within a changed setting range, and uses an image obtained by processing the cross-sectional image for updating the three-dimensional image when the pulsation score is outside the changed setting range.

[6] The image processing device according to [4], in which the control unit sequentially changes the setting range, and every time the setting range is changed, for each position in the movement direction of the sensor, the control unit uses an acquired cross-sectional image for updating the three-dimensional image when a derived pulsation score is within a changed setting range, and uses an image obtained by processing the cross-sectional image for updating the three-dimensional image when the pulsation score is outside the changed setting range.

[7] The image processing device according to any one of [4] to [6], in which for each position in the movement direction of the sensor, the control unit inputs an acquired cross-sectional image and the setting range to a learned model, and obtains a processed cross-sectional image output from the learned model as the processed image when a derived pulsation score is outside the setting range.

[8] The image processing device according to [7], in which the control unit uses, as the learned model, a model generated or updated by a module that uses artificial intelligence.

[9] An image display system includes: the image processing device according to any one of [2] to [8]; and a display configured to display the three-dimensional image.

[10] An image processing device includes: a control unit configured to acquire a cross-sectional image obtained using a sensor configured to move in a lumen of a biological tissue and a pulsation score representing expansion and contraction of the lumen in the cross-sectional image by a numerical value for each position in a movement direction of the sensor, select the cross-sectional image when the acquired pulsation score is within a setting range, process the cross-sectional image when the pulsation score is outside the setting range, and generate a three-dimensional image representing the biological tissue based on a selected image group and a processed image group obtained for a plurality of positions in the movement direction of the sensor.

[11] The image processing device according to [10], in which when an operation of changing the setting range is received, for each position in the movement direction of the sensor, the control unit uses an acquired cross-sectional image for updating the three-dimensional image when an acquired pulsation score is within a changed setting range, and uses an image obtained by processing the cross-sectional image for updating the three-dimensional image when the pulsation score is outside the changed setting range.

[12] The image processing device according to [10], in which the control unit sequentially changes the setting range, and every time the setting range is changed, for each position in the movement direction of the sensor, the control unit uses an acquired cross-sectional image for updating the three-dimensional image when an acquired pulsation score is within a changed setting range, and uses an image obtained by processing the cross-sectional image for updating the three-dimensional image when the pulsation score is outside the changed setting range.

[13] The image processing device according to any one of to [12], in which for each position in the movement direction of the sensor, the control unit inputs an acquired cross-sectional image and the setting range to a learned model, and obtains a processed cross-sectional image output from the learned model as a processed image when an acquired pulsation score is outside the setting range.

[14] The image processing device according to [13], in which the control unit uses, as the learned model, a model generated or updated by a module that uses artificial intelligence.

[15] An image display system includes: the image processing device according to any one of [10] to [14]; and a display configured to display the three-dimensional image.

[16] An image processing device includes: a control unit configured to acquire a plurality of cross-sectional images that are obtained at time points different from one another by using a sensor configured to move in a lumen of a biological tissue and that have different pulsation scores representing expansion and contraction of the lumen in the cross-sectional images by a numerical value for each position in a movement direction of the sensor, select a cross-sectional image whose pulsation score is within a setting range among the plurality of acquired cross-sectional images, and generate a three-dimensional image representing the biological tissue based on a selected image group obtained for a plurality of positions in the movement direction of the sensor.

[17] The image processing device according to [16], in which when an operation of changing the setting range is received, for each position in the movement direction of the sensor, the control unit uses a cross-sectional image whose pulsation score is within a changed setting range among the plurality of acquired cross-sectional images for updating the three-dimensional image.

[18] The image processing device according to [16], in which the control unit sequentially changes the setting range, and every time the setting range is changed, for each position in the movement direction of the sensor, the control unit uses a cross-sectional image whose pulsation score is within a changed setting range among the plurality of acquired cross-sectional images for updating the three-dimensional image.

[19] An image processing method comprising: acquiring a cross-sectional image obtained using a sensor configured to move in a lumen of a biological tissue for each position in a movement direction of the sensor; analyzing the acquired cross-sectional image to calculate a centroid position of a cross section of the lumen in the cross-sectional image; and executing smoothing on a calculation result obtained for a plurality of positions in the movement direction of the sensor; evaluating a difference between the calculated centroid position and a centroid position obtained as a result of the smoothing for each position in the movement direction of the sensor; and deriving a pulsation score representing expansion and contraction of the lumen in the acquired cross-sectional image by a numerical value.

[20] A non-transitory computer-readable medium storing an image processing program that causes a computer to execute a process comprising: acquiring a cross-sectional image obtained using a sensor configured to move in a lumen of a biological tissue for each position in a movement direction of the sensor; analyzing the acquired cross-sectional image to calculate a centroid position of a cross section of the lumen in the cross-sectional image; executing smoothing on a calculation result obtained for a plurality of positions in the movement direction of the sensor; evaluating a difference between the calculated centroid position and a centroid position obtained as a result of the smoothing for each position in the movement direction of the sensor; and deriving a pulsation score representing expansion and contraction of the lumen in the acquired cross-sectional image by a numerical value.

According to the present disclosure, it is possible to reduce an influence of a pulsation when generating a three-dimensional image representing a biological tissue, or provide information indicating a timing in a pulsation cycle at which a two-dimensional image that can be used for generating such a three-dimensional image is acquired.

### BRIEF DESCRIPTION OF THE DRAWINGS

FIG. 1 is a perspective view of an image display system according to an embodiment of the present disclosure.

FIG. 2 is a perspective view of a probe and a drive unit according to the embodiment of the present disclosure.

FIG. 3 is a diagram showing an example of a cross-sectional image, a selected image, and a pulsation score according to the embodiment of the present disclosure.

FIG. 4 is a diagram showing an example of a cross-sectional image obtained using a sensor according to the embodiment of the present disclosure and a cross-sectional image after classification performed by a learned model.

FIG. 5 is a block diagram showing a configuration of an image processing device according to the embodiment of the present disclosure.

FIG. 6 is a flowchart showing an operation of an image display system according to the embodiment of the present disclosure.

FIG. 7 is a flowchart showing a specific example of the operation of the image display system according to the embodiment of the present disclosure.

FIG. 8 is a flowchart showing a first modification of the operation of the image display system according to the embodiment of the present disclosure.

FIG. 9 is a diagram showing an example of a cross-sectional image obtained using the sensor according to the embodiment of the present disclosure and a cross-sectional image after processing performed by the learned model.

FIG. 10 is a flowchart showing a second modification of the operation of the image display system according to the embodiment of the present disclosure.

FIG. 11 is a diagram showing an example of a plurality of cross-sectional images and a selected image according to the embodiment of the present disclosure.

FIG. 12 is a flowchart showing a third modification of the operation of the image display system according to the embodiment of the present disclosure.

FIG. 13 is a flowchart showing a fourth modification of the operation of the image display system according to the embodiment of the present disclosure.

FIG. 14 is a flowchart showing a fifth modification of the operation of the image display system according to the embodiment of the present disclosure.

### DETAILED DESCRIPTION

Set forth below with reference to the accompanying drawings is a detailed description of embodiments of image display system, an image processing method, and an image processing program.

In the drawings, the same or corresponding parts are denoted by the same reference numerals. In description of the present embodiment, description of the same or corresponding parts will be omitted or simplified as appropriate.

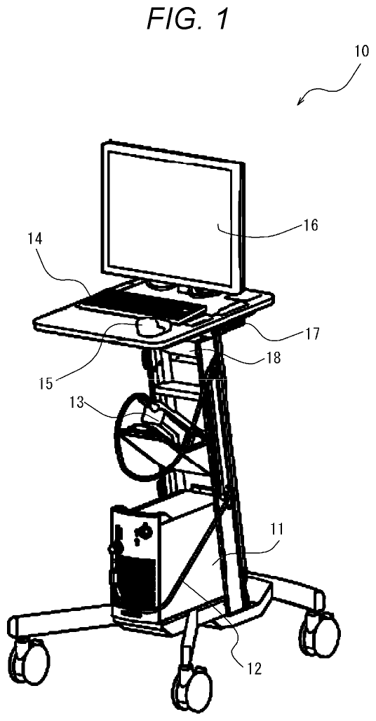

A configuration of an image display system 10 according to the present embodiment will be described with reference to FIG. 1.

The image display system 10 includes an image processing device 11, a cable 12, a drive unit 13, a keyboard 14, a mouse 15, and a display 16.

The image processing device 11 is a dedicated computer specialized for image diagnosis in the present embodiment, but may be a general-purpose computer such as a personal computer (PC).

The cable 12 is used to connect the image processing device 11 to the drive unit 13.

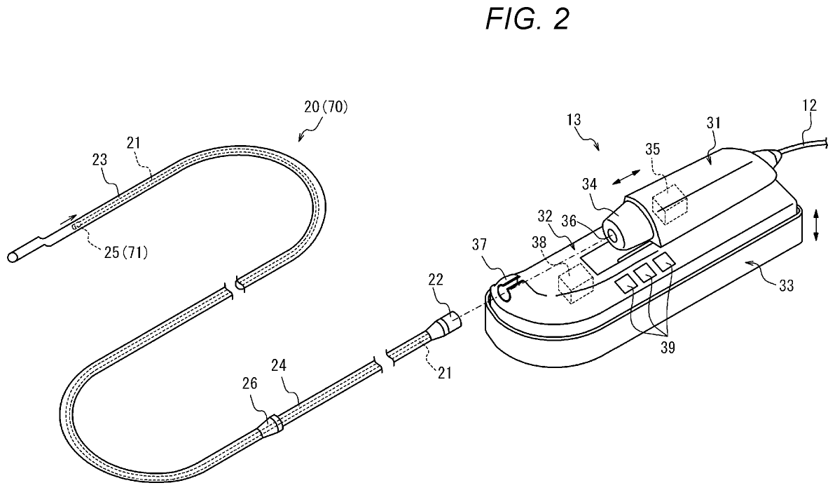

The drive unit 13 is a device that is used by being connected to a probe 20 shown in FIG. 2, and that drives the probe 20. The drive unit 13 is also referred to as a motor drive unit (MDU). The probe 20 is applied to IVUS. The probe 20 is also referred to as an IVUS catheter or an image diagnosis catheter.

The keyboard 14, the mouse 15, and the display 16 are connected to the image processing device 11 via any cable or in a wireless manner. The display 16 can be, for example, a liquid crystal display (LCD), an organic electro luminescent (EL) display, or a head-mounted display (HMD).

The image display system 10 can further include a connection terminal 17 and a cart unit 18 as options.

The connection terminal 17 is used to connect the image processing device 11 to an external device. The connection terminal 17 can be, for example, a universal serial bus (USB) terminal. The external device can be, for example, a recording medium such as a magnetic disk drive, a magneto-optical disk drive, or an optical disk drive.

The cart unit 18 can be a cart with casters for movement. The image processing device 11, the cable 12, and the drive unit 13 can be installed in a cart body of the cart unit 18. The keyboard 14, the mouse 15, and the display 16 can be installed on an uppermost table of the cart unit 18.

Configurations of the probe 20 and the drive unit 13 according to the present embodiment will be described with reference to FIG. 2.

The probe 20 can include a drive shaft 21, a hub 22, a sheath 23, an outer tube 24, an ultrasound transducer 25, and a relay connector 26.

The drive shaft 21 passes through the sheath 23 inserted into a lumen in a living body and the outer tube 24 connected to a proximal end of the sheath 23, and extends to inside of the hub 22 provided at a proximal end of the probe 20. The drive shaft 21 can include the ultrasound transducer 25, which transmits and receives a signal, at a distal end of the drive shaft 21, and is rotatably provided in the sheath 23 and the outer tube 24. The relay connector 26 connects the sheath 23 to the outer tube 24.

The hub 22, the drive shaft 21, and the ultrasound transducer 25 are connected to one another so as to integrally move forward and backward in an axial direction. Therefore, for example, when the hub 22 is pressed toward a distal side, the drive shaft 21 and the ultrasound transducer 25 move toward the distal side in the sheath 23 in a direction opposite to a direction indicated by an arrow. For example, when the hub 22 is pulled toward a proximal side, the drive shaft 21 and the ultrasound transducer 25 move toward the proximal side in the sheath 23 in the direction indicated by the arrow.

The drive unit 13 can include a scanner unit 31, a slide unit 32, and a bottom cover 33.

The scanner unit 31 is also referred to as a pullback unit. The scanner unit 31 is connected to the image processing device 11 via the cable 12. The scanner unit 31 includes a probe connection section 34 connected to the probe 20, and a scanner motor 35 that is a drive source that rotates the drive shaft 21.

The probe connection section 34 is freely detachably connected to the probe 20 via an insertion port 36 of the hub 22 provided at the proximal end of the probe 20. In the hub 22, a proximal end of the drive shaft 21 is rotatably supported, and a rotational force of the scanner motor 35 is transmitted to the drive shaft 21. Further, a signal is transmitted and received between the drive shaft 21 and the image processing device 11 via the cable 12. In the image processing device 11, a tomographic image of a biological lumen is generated and an image is processed based on a signal transmitted from the drive shaft 21.

The slide unit 32 mounts the scanner unit 31 so as to be movable forward and backward, and is mechanically and electrically connected to the scanner unit 31. The slide unit 32 can include a probe clamp portion 37, a slide motor 38, and a switch group 39.

The probe clamp portion 37 is co-axially disposed with the probe connection section 34 on a distal side from the probe connection section 34, and supports the probe 20 connected to the probe connection section 34.

The slide motor 38 is a drive source that generates a driving force in an axial direction. The scanner unit 31 moves forward or backward by driving of the slide motor 38, and the drive shaft 21 moves forward or backward in an axial direction accordingly. The slide motor 38 can be, for example, a servomotor.

The switch group 39 can include, for example, a forward switch and a pull-back switch pressed during a forward and backward operation of the scanner unit 31, and a scan switch pressed when image drawing is started or ended. The present disclosure is not limited to the example here, and various switches are included in the switch group 39 when necessary.

When the forward switch is pressed, the slide motor 38 is rotated in a forward direction, and the scanner unit 31 advances. On the other hand, when the pull-back switch is pressed, the slide motor 38 is rotated in a reverse direction, and the scanner unit 31 retracts.

When the scan switch is pressed, the image drawing is started, the scanner motor 35 is driven, and the slide motor 38 is driven to repeat advance and retract of the scanner unit 31. A user such as a surgeon connects the probe 20 to the scanner unit 31 in advance, the image drawing is started, and the drive shaft 21 repeats movement toward a distal side in an axial direction and movement toward a proximal side in the axial direction while being rotated. When the scan switch is pressed again, the scanner motor 35 and the slide motor 38 are stopped, and the image drawing is ended.

The bottom cover 33 covers a bottom and an entire periphery of a side surface on a bottom side of the slide unit 32, and is movable toward and away from the bottom of the slide unit 32.

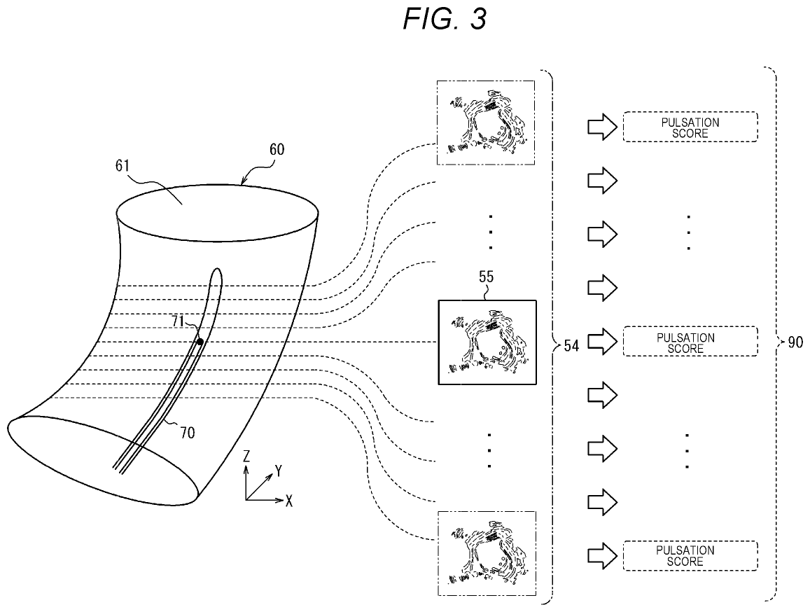

An outline of the present embodiment will be described with reference to FIGS. 3 to 5.

The image processing device 11 acquires a cross-sectional image 54 obtained using a sensor 71 for each position in a movement direction of the sensor 71 that moves in a lumen 61 of a biological tissue 60. When the sensor 71 is the ultrasound transducer 25, the sensor 71 repeats transmission and reception of ultrasound signals while being rotated, and one (one frame) cross-sectional image 54 includes hundreds items (or pieces) of line data obtained by one-time ultrasound transmission and reception. The image processing device 11 analyzes the acquired cross-sectional image 54 for each position in the movement direction of the sensor 71 to calculate a centroid position of a cross section of the lumen 61 in the cross-sectional image 54. The image processing device 11 executes smoothing on calculation results obtained for a plurality of positions in the movement direction of the sensor 71. The smoothing can be executed by, for example, moving-averaging an X coordinate value of the cross-sectional image 54 of the calculated centroid position for a movement distance corresponding to at least time of one cycle of a pulsation in the movement direction of the sensor 71 in a two-dimensional coordinate system in which an X-axis of the acquired cross-sectional image 54 is a vertical axis and the movement direction of the sensor 71 is a horizontal axis. The image processing device 11 evaluates a difference between the calculated actual centroid position of the cross-sectional image 54 (for example, the X coordinate value) and the centroid position obtained as a result of the smoothing (for example, the X coordinate value) for each position in the movement direction of the sensor 71, thereby deriving a pulsation score 90 representing expansion and contraction of the lumen 61 in the acquired cross-sectional image 54 by using a numerical value. The pulsation score 90 is a natural number in the present embodiment, and may be an integer or a real number. The display 16 may display the pulsation score 90 derived by the image processing device 11 for each position in the movement direction of the sensor 71. For example, the pulsation non-uniformly deforms a lumen wall that defines the lumen 61 where the probe 20 is disposed in a radial direction centered around the centroid position, and cyclically generates substantially the same deformation (expansion or contraction). Therefore, the centroid position in the cross-sectional image of the lumen 61 varies every time in one cycle of the pulsation. On the other hand, the centroid position obtained as a result of the smoothing is regarded as a centroid position in an intermediate period in one cycle of the pulsation, that is, in an intermediate state where the lumen 61 neither expands nor contracts. Therefore, by evaluating the difference between the calculated actual centroid position of the cross-sectional image 54 and the centroid position obtained as a result of the smoothing for each position in the movement direction of the sensor 71, it is possible to derive the pulsation score 90 representing the expansion and contraction of the lumen 61 in the acquired cross-sectional image 54 by using a numerical value. Note that the pulsation score 90 may be derived by evaluating a difference between a Y coordinate value of the cross-sectional image 54 of the calculated centroid position and a result obtained by smoothing the Y coordinate values at the plurality of positions in the movement direction of the sensor 71. Further, the pulsation score 90 may be derived by evaluating a difference between XY coordinate value of the cross-sectional image 54 of the calculated centroid position and a result obtained by smoothing the XY coordinate values at the plurality of positions in the movement direction of the sensor 71. In this case, the smoothing can be executed by three-dimensionally moving-averaging the XY coordinate value of the cross-sectional image 54 of the calculated centroid position for a movement distance corresponding to at least time of one cycle of the pulsation in the movement direction of the sensor 71 in a three-dimensional coordinate system in which an X coordinate of the acquired cross-sectional image 54 is a first axis, a Y coordinate of the acquired cross-sectional image 54 is a second axis, and the movement direction of the sensor 71 is a third axis.

The image processing device 11 acquires a plurality of cross-sectional images 54 and a plurality of pulsation scores 90 for the positions in the movement direction of the sensor 71 by repeating a plurality of times processing of the cross-sectional image acquisition and the pulsation score acquisition in an axial direction in the lumen in the living body described above. Next, the image processing device 11 determines which of the plurality of acquired cross-sectional images 54 is to select according to the plurality of derived pulsation scores 90 for the positions in the movement direction of the sensor 71. The image processing device 11 generates a three-dimensional image 53 representing the biological tissue 60 based on a selected image group obtained for at least some of positions among the plurality of positions in the movement direction of the sensor 71. The display 16 displays the three-dimensional image 53 generated by the image processing device 11. Note that after one-time cross-sectional image acquisition processing, it is also possible to evaluate the pulsation scores 90, select only images whose pulsation scores 90 satisfy a condition, and generate the three-dimensional image 53 without using any other images.

According to the present embodiment, the two-dimensional image group used for generating the three-dimensional image 53 is selected according to the pulsation scores 90 representing the expansion and contraction of the lumen 61 in the two-dimensional images by using numerical values, so that it is relatively easy to select a two-dimensional image group acquired at the same timings in the pulsation cycle. As a result, it is possible to reduce an influence of the pulsation when generating the three-dimensional image 53. That is, it is possible to generate an image with almost no unevenness due to the influence of the pulsation as the three-dimensional image 53. Therefore, it is relatively easy for the surgeon to accurately grasp a structure of the biological tissue 60 based on the three-dimensional image 53.

According to the present embodiment, the pulsation score 90 is derived for the two-dimensional image that can be used for generating the three-dimensional image 53, whereby it is also possible to provide information indicating a timing in the pulsation cycle at which the two-dimensional image is acquired. For example, the pulsation score 90 is displayed for a certain two-dimensional image, whereby it is possible to notify the user such as the surgeon whether the two-dimensional image is an image acquired during the expansion of the lumen 61 or an image acquired during the contraction of the lumen 61. Alternatively, another device generates the three-dimensional image 53 instead of generating the three-dimensional image 53 by the image processing device 11, and the image processing device 11 notifies the other device of the pulsation score 90, whereby the other device can generate an image with almost no unevenness due to the influence of the pulsation as the three-dimensional image 53.

The biological tissue 60 can include, for example, a blood vessel or an organ such as a heart. The biological tissue 60 is not only limited to an anatomical single organ or a part of the anatomical single organ, and also includes a tissue having a lumen across a plurality of organs. Examples of such a tissue include, specifically, a part of a vascular tissue from an upper portion of an inferior vena cava through a right atrium to a lower portion of a superior vena cava.

In the present embodiment, the image processing device 11 refers to tomographic data 51 that is a data set of the cross-sectional image 54 obtained using the sensor 71, and generates and updates three-dimensional data 52 indicating the biological tissue 60. The image processing device 11 displays the three-dimensional data 52 on the display 16 as the three-dimensional image 53. That is, the image processing device 11 refers to the tomographic data 51, and displays the three-dimensional image 53 on the display 16.

The image processing device 11 may form, in the three-dimensional data 52, an opening that exposes the lumen 61 of the biological tissue 60 in the three-dimensional image 53. The image processing device 11 may adjust a viewpoint when displaying the three-dimensional image 53 on a screen according to a position of the opening. The viewpoint refers to a position of a virtual camera disposed in a three-dimensional space.

The sensor 71 is provided in a catheter 70. The catheter 70 is the probe 20, that is, the IVUS catheter in the present embodiment, and may be an optical frequency domain imaging (OFDI) catheter or an optical coherence tomography (OCT) catheter. That is, the sensor 71 is the ultrasound transducer 25 in the present embodiment, and transmits ultrasounds in the lumen 61 of the biological tissue 60 to acquire the tomographic data 51. Alternatively, the sensor 71 may radiate light in the lumen 61 of the biological tissue 60 to acquire the tomographic data 51. When emitting light, it is conceivable to provide a distal portion of an optical fiber as a transmitting and receiving unit that performs light emission and light reception at a distal end of the catheter 70.

In FIG. 3, a Z direction corresponds to the movement direction of the sensor 71, but for the sake of convenience, the Z direction may be regarded as a direction corresponding to a longitudinal direction of the lumen 61 of the biological tissue 60. An X direction orthogonal to the Z direction and a Y direction orthogonal to the Z direction and the X direction may be regarded as directions corresponding to a transverse direction of the lumen 61 of the biological tissue 60.

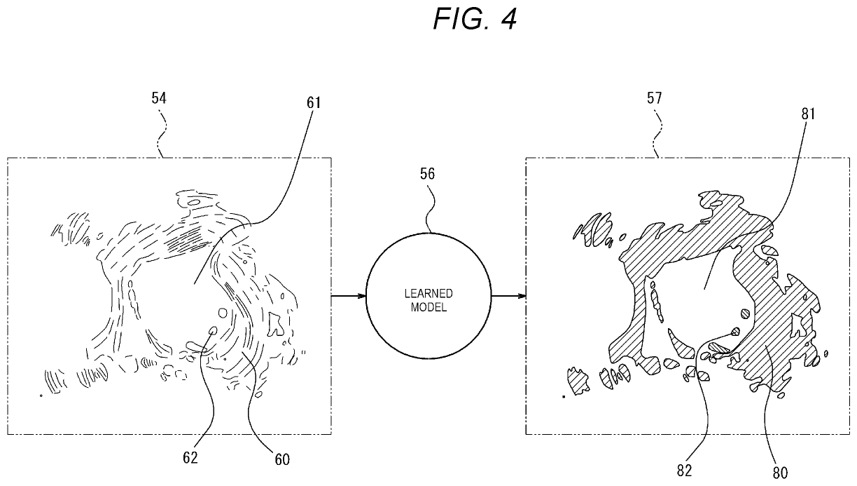

In the present embodiment, every time the IVUS two-dimensional image serving as the cross-sectional image 54 is obtained, the image processing device 11 detects a pixel group included in the obtained two-dimensional image and representing the lumen 61 as a cross section of the lumen 61. Pixels representing the lumen 61 are identified using, for example, a learned model 56 as shown in FIG. 4. The image processing device 11 calculates a centroid position of the detected cross section. As a method for calculating the centroid position, for example, a method similar to that disclosed in International Patent Application Publication No. WO 2021/200294 is used. The image processing device 11 executes the smoothing on calculation results of centroid positions of a plurality of cross sections present along the Z direction. As a method for executing the smoothing, for example, a method similar to that disclosed in International Patent Application Publication No. 2021/200294 is used. The image processing device 11 calculates, as the pulsation score 90, a distance between the calculated centroid position and the centroid position obtained as a result of the smoothing for the obtained two-dimensional image. When the calculated pulsation score 90 is in a predetermined range or coincides with a predetermined value, the image processing device 11 selects the two-dimensional image as a cross-sectional image 55 used for generating or updating the three-dimensional image 53. The image processing device 11 uses the selected cross-sectional image 55 to draw the three-dimensional image 53.

In the present embodiment, the image processing device 11 uses the learned model 56 to classify the plurality of pixels included in each cross-sectional image 54 into two or more classes. The two or more classes can include a biological tissue class 80 corresponding to pixels representing the biological tissue 60, a blood cell class 81 corresponding to pixels representing blood cells contained in blood that flows through the lumen 61, and a medical instrument class 82 corresponding to pixels representing a medical instrument 62 inserted into the lumen 61 such as a catheter other than the catheter 70 or a guide wire. An indwelling object class corresponding to pixels representing an indwelling object such as a stent may be further provided. A lesion class corresponding to pixels representing a lesion such as lime or plaque may be further provided. Each class may be subdivided. For example, the medical instrument class 82 may be divided into a catheter class, a guide wire class, and other medical instrument classes.

The learned model 56 is trained to be able to detect a region corresponding to each class based on the two-dimensional image that is a sample by performing machine learning in advance. When the cross-sectional image 54 is input, the learned model 56 outputs, as a classification result, a cross-sectional image 57 in which a classification of any one of the biological tissue class 80, the blood cell class 81, and the medical instrument class 82 is given to pixels of the input cross-sectional image 54. In the present embodiment, pixels classified into the blood cell class 81 are treated as the pixels representing the lumen 61, and pixels classified into classes other than the biological tissue class 80 such as pixels classified into the medical instrument class 82 may be further treated as the pixels representing the lumen 61.

In the present embodiment, the image processing device 11 generates the three-dimensional image 53 by stacking a pixel group included in an image group selected as the cross-sectional images 55 and classified into the biological tissue class 80, and making the pixel group three-dimensional. The image processing device 11 may further generate a three-dimensional image of the medical instrument 62 by stacking a pixel group included in the image group selected as the cross-sectional images 55 and classified into the medical instrument class 82, and making the pixel group three-dimensional. Alternatively, the image processing device 11 may further generate, as the three-dimensional image of the medical instrument 62, a linear three-dimensional model that connects coordinates of the pixel group included in the image group selected as the cross-sectional images 55 and classified into the medical instrument class 82.

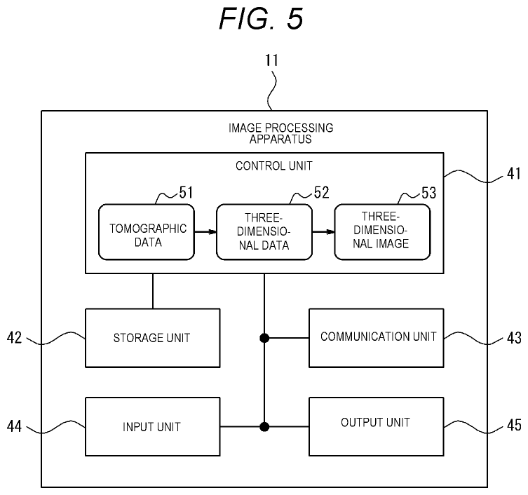

A configuration of the image processing device 11 will be described with reference to FIG. 5.

The image processing device 11 can include a control unit 41, a storage unit 42, a communication unit 43, an input unit 44, and an output unit 45.

The control unit 41 can include at least one processor, at least one programmable circuit, at least one dedicated circuit, or any combination of the at least one processor, the at least one programmable circuit, and the at least one dedicated circuit. The processor can be a general-purpose processor, for example, such as a central processing unit (CPU) or a graphics processing unit (GPU), or a dedicated processor specialized for specific processing. The programmable circuit can be, for example, a field-programmable gate array (FPGA). The dedicated circuit can be, for example, an application specific integrated circuit (ASIC). The control unit 41 executes processing related to an operation of the image processing device 11 while controlling units of the image display system 10 including the image processing device 11.

The storage unit 42 can include at least one semiconductor memory, at least one magnetic memory, at least one optical memory, or any combination of the at least one semiconductor memory, the at least one magnetic memory, and the at least one optical memory. The semiconductor memory can be, for example, a random access memory (RAM) or a read only memory (ROM). The RAM can be, for example, a static random access memory (SRAM) or a dynamic random access memory (DRAM). The ROM can be, for example, an electrically erasable programmable read only memory (EEPROM). The storage unit 42 can function as, for example, a main storage device, an auxiliary storage device, or a cache memory. Data used for the operation of the image processing device 11 such as the tomographic data 51, and data obtained by the operation of the image processing device 11 such as the three-dimensional data 52 and the three-dimensional image 53 are stored in the storage unit 42. A region for storing the cross-sectional images 54 and 55 shown in FIG. 3 is set in the storage unit 42.

The communication unit 43 can include at least one communication interface. The communication interface can be, for example, a wired local area network (LAN) interface corresponding to a communication standard such as Ethernet®, a wireless LAN interface corresponding to a communication standard such as Institute of Electrical and Electronics Engineers 802.11 (IEEE 802.11), or an image diagnosis interface that performs an analog to digital (A/D) conversion on the received IVUS signal. The communication unit 43 receives the data used for the operation of the image processing device 11, and transmits the data obtained by the operation of the image processing device 11. In the present embodiment, the drive unit 13 is connected to the image diagnosis interface provided in the communication unit 43.

The input unit 44 can include at least one input interface. The input interface cam be, for example, a USB interface, a high-definition multimedia interface (HDMI®) interface, or an interface corresponding to a short-range wireless communication standard such as Bluetooth®. The input unit 44 receives an operation of the user such as an operation of inputting the data used for the operation of the image processing device 11. In the present embodiment, the keyboard 14 and the mouse 15 are connected to the USB interface or the interface corresponding to the short-range wireless communication provided in the input unit 44. When a touchscreen is provided integrally with the display 16, the display 16 may be connected to the USB interface or the HDMI interface provided in the input unit 44.

The output unit 45 can include at least one output interface. The output interface can be, for example, the USB interface, the HDMI interface, or the interface corresponding to the short-range wireless communication standard such as Bluetooth. The output unit 45 outputs data obtained by the operation of the image processing device 11. In the present embodiment, the display 16 is connected to the USB interface or the HDMI interface provided in the output unit 45.

Functions of the image processing device 11 are implemented by executing an image processing program according to the present embodiment by a processor serving as the control unit 41. That is, the functions of the image processing device 11 are implemented by software. The image processing program causes a computer to function as the image processing device 11 by causing the computer to execute the operation of the image processing device 11. That is, the computer functions as the image processing device 11 by executing the operation of the image processing device 11 according to the image processing program.

The program can be stored in a non-transitory computer-readable medium in advance. The non-transitory computer-readable medium can be, for example, a flash memory, a magnetic recording device, an optical disk, a magneto-optical recording medium, or a ROM. The program is distributed by, for example, selling, transferring, or lending a portable medium such as a secure digital (SD) card, a digital versatile disc (DVD), or a compact disc read only memory (CD-ROM) where the program is stored. The program may be distributed by storing the program in a storage of a server in advance and transferring the program from the server to another computer. The program may be provided as a program product.

The computer temporarily stores, for example, the program stored in the portable medium or the program transferred from the server in the main storage device. The computer reads the program stored in the main storage device by the processor, and executes processing according to the read program by the processor. The computer may read the program directly from the portable medium, and execute the processing according to the program. Every time the program is transferred from the server to the computer, the computer may sequentially execute the processing according to the received program. The processing may be executed by a so-called application service provider (ASP) service in which a function is implemented only by an execution instruction and result acquisition without transferring the program from the server to the computer. The program includes information provided for processing performed by an electronic computer and conforming to the program. For example, data that is not a direct command to the computer but has a property of defining processing of the computer corresponds to the “information conforming to the program”.

Some or all functions of the image processing device 11 may be implemented by the programmable circuit or the dedicated circuit serving as the control unit 41. That is, some or all functions of the image processing device 11 may be implemented by hardware.

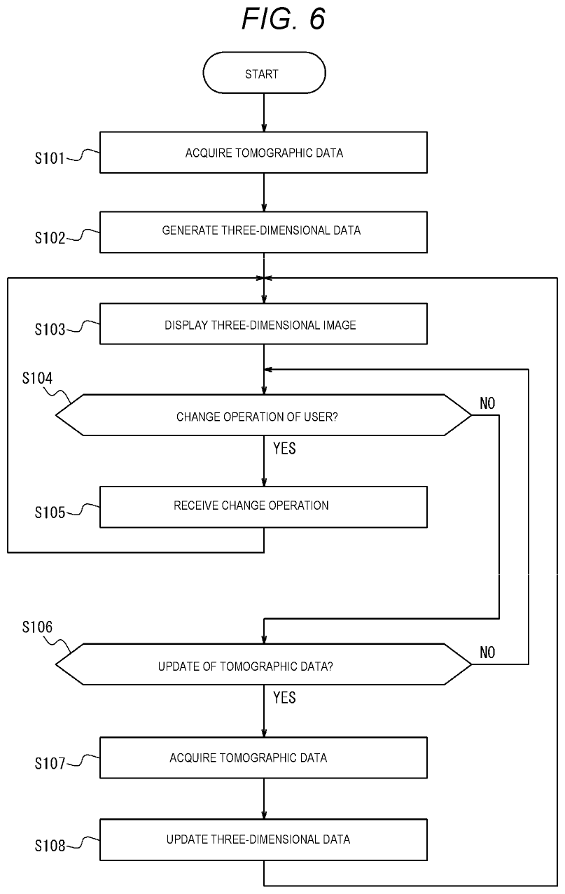

An operation of the image display system 10 according to the present embodiment will be described with reference to FIG. 6. The operation of the image display system 10 corresponds to an image processing method according to the present embodiment.

Before starting a flow in FIG. 6, the probe 20 is primed by the user.

Thereafter, the probe 20 is fitted into the probe connection section 34 and the probe clamp portion 37 of the drive unit 13, and is connected and fixed to the drive unit 13. The probe 20 is inserted to a target area in the biological tissue 60 such as the blood vessel or the heart.

In S101, when the scan switch provided in the switch group 39 is pressed and the pull-back switch provided in the switch group 39 is pressed, a so-called pull-back operation is performed. The probe 20 transmits ultrasounds by the ultrasound transducer 25 that retracts in the axial direction by the pull-back operation in the biological tissue 60. The ultrasound transducer 25 radially transmits the ultrasounds while moving in the biological tissue 60. The ultrasound transducer 25 receives reflected waves of the transmitted ultrasounds. The probe 20 inputs signals of the reflected waves received by the ultrasound transducer 25 to the image processing device 11. The control unit 41 of the image processing device 11 processes the input signals to sequentially generate the cross-sectional images 54 of the biological tissue 60, thereby acquiring the tomographic data 51 including the plurality of cross-sectional images 54.

Specifically, the probe 20 transmits the ultrasounds in a plurality of directions from a rotation center toward an outer side by the ultrasound transducer 25 while rotating the ultrasound transducer 25 in a circumferential direction and moving the ultrasound transducer 25 in the axial direction in the biological tissue 60. The probe 20 receives a reflected wave from a reflection object present in each of the plurality of directions in the biological tissue 60 by the ultrasound transducer 25. The probe 20 transmits the signals of the received reflected waves to the image processing device 11 via the drive unit 13 and the cable 12. The communication unit 43 of the image processing device 11 receives the signals transmitted from the probe 20. The communication unit 43 performs A/D conversion on the received signals. The communication unit 43 inputs the A/D converted signals to the control unit 41. The control unit 41 processes the input signals to calculate an intensity value distribution of the reflected waves from a reflection object present in a transmission direction of the ultrasounds of the ultrasound transducer 25. The control unit 41 sequentially generates two-dimensional images having a luminance value distribution corresponding to the calculated intensity value distribution as the cross-sectional images 54 of the biological tissue 60, thereby acquiring the tomographic data 51 that is the data set of the cross-sectional images 54. The control unit 41 stores the acquired tomographic data 51 in the storage unit 42.

In the present embodiment, the signals of the reflected waves received by the ultrasound transducer 25 correspond to raw data of the tomographic data 51, and the cross-sectional images 54 generated by processing the signals of the reflected waves by the image processing device 11 correspond to processing data of the tomographic data 51.

As a modification of the present embodiment, the control unit 41 of the image processing device 11 may store the signals input from the probe 20 as the tomographic data 51 as they are in the storage unit 42. Alternatively, the control unit 41 may store, as the tomographic data 51, data indicating the intensity value distribution of the reflected waves calculated by processing the signals input from the probe 20 in the storage unit 42. That is, the tomographic data 51 is not limited to the data set of the cross-sectional images 54 of the biological tissue 60, and may be data representing cross sections of the biological tissue 60 at movement positions of the ultrasound transducer 25 in some form.

As a modification of the present embodiment, a so-called array type in which a plurality of ultrasound transducers are arranged in a circumferential direction is used instead of using the ultrasound transducer 25 that transmits the ultrasounds in a plurality of directions while being rotated in the circumferential direction, whereby the ultrasounds may be transmitted in the plurality of directions without rotation.

As a modification of the present embodiment, instead of generating the data set of the cross-sectional images 54 of the biological tissue 60 by the image processing device 11, another device may generate a similar data set, and the image processing device 11 may acquire the data set from the other device. That is, instead of generating the cross-sectional images 54 of the biological tissue 60 by processing the IVUS signals by the control unit 41 of the image processing device 11, another device may generate the cross-sectional images 54 of the biological tissue 60 by processing the IVUS signals, and input the generated cross-sectional images 54 to the image processing device 11.

In S102, the control unit 41 of the image processing device 11 generates the three-dimensional data 52 of the biological tissue 60 based on the tomographic data 51 acquired in S101. That is, the control unit 41 generates the three-dimensional data 52 based on the tomographic data 51 acquired by the sensor 71. Here, when the already generated three-dimensional data 52 is present, it is preferable to only update data at a location corresponding to the updated tomographic data 51, instead of regenerating all items (or pieces) of three-dimensional data 52 from the beginning. In this case, a data processing amount when generating the three-dimensional data 52 can be reduced, and a real-time property of the three-dimensional image 53 in subsequent S103 can be improved.

Specifically, the control unit 41 of the image processing device 11 analyzes the cross-sectional image 54 of the biological tissue 60 included in the tomographic data 51 stored in the storage unit 42 for each position in the movement direction of the ultrasound transducer 25 serving as the sensor 71 to calculate the centroid position of the cross section of the lumen 61 in the cross-sectional image 54. The control unit 41 executes the smoothing on calculation results obtained for a plurality of positions in the movement direction of the ultrasound transducer 25. The control unit 41 evaluates the difference between the calculated centroid position and the centroid position obtained as a result of the smoothing for each position in the movement direction of the ultrasound transducer 25, thereby deriving the pulsation score 90 representing the expansion and contraction of the lumen 61 in the cross-sectional image 54 by a numerical value. The control unit 41 determines whether to select the cross-sectional image 54 according to the derived pulsation score 90 for each position in the movement direction of the ultrasound transducer 25. More specifically, when the derived pulsation score 90 is within a setting range, the control unit 41 selects the cross-sectional image 54 as the cross-sectional image 55 used for generating the three-dimensional image 53. On the other hand, when the pulsation score 90 is outside the setting range, the control unit 41 does not select the cross-sectional image 54. The setting range may be a numerical value range having a certain width (or breadth) or may be one numerical value. The control unit 41 stacks the selected image group obtained for at least some positions among the plurality of positions in the movement direction of the ultrasound transducer 25 and makes the selected image group three-dimensional, thereby generating the three-dimensional data 52 of the biological tissue 60. As the three-dimensional methods, a rendering method such as a surface rendering or a volume rendering, and any accompanying method in various processing such as texture mapping including environment mapping and bump mapping are used. The control unit 41 stores the generated three-dimensional data 52 in the storage unit 42.

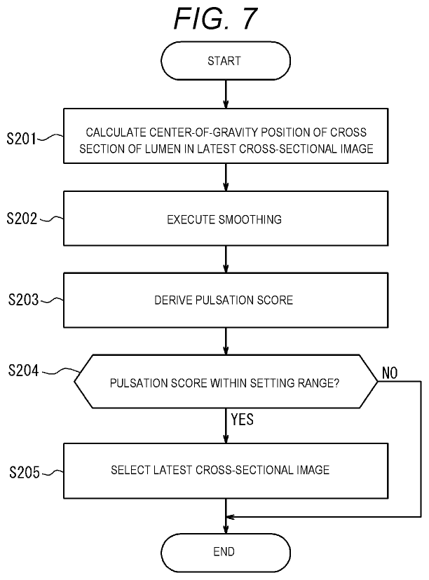

FIG. 7 shows a specific example of an operation of selecting the cross-sectional image 55 when the latest cross-sectional image 54 is acquired. In S201 shown in FIG. 7, the control unit 41 of the image processing device 11 calculates a centroid position of a cross section of the lumen 61 in the latest cross-sectional image 54. In S202, the control unit 41 executes the smoothing on calculation results of centroid positions of cross sections of the lumen 61 in a plurality of cross-sectional images 54 including the latest cross-sectional image 54 and one or more other cross-sectional images 54 obtained when the ultrasound transducer 25 is at other positions before time at which the latest cross-sectional image 54 is obtained. For the processing in S201 and S202, for example, a method similar to that disclosed in International Patent Application Publication No. WO 2021/200294 can be used. In S203, the control unit 41 calculates, as the pulsation score 90, a distance between the centroid position calculated in S201 and the centroid position obtained as a result of the smoothing in S202. In S204, the control unit 41 determines whether the pulsation score 90 calculated in S203 is within the setting range. When it is determined in S204 that the pulsation score 90 is within the setting range, the control unit 41 selects the latest cross-sectional image 54 as the cross-sectional image 55 in S205. When it is determined in S204 that the pulsation score 90 is outside the setting range, the operation shown in FIG. 7 is ended.

The control unit 41 of the image processing device 11 classifies the pixel group of the cross-sectional images 54 included in the tomographic data 51 acquired in S101 into two or more classes. The two or more classes include the biological tissue class 80, the blood cell class 81, and the medical instrument class 82, and may further include the indwelling object class or the lesion class. As a classification method, any method may be used. In the present embodiment, as shown in FIG. 4, a method for classifying the pixel group of the cross-sectional images 54 by the learned model 56 is used. Therefore, in S201, the control unit 41 detects the pixel group representing the lumen 61 such as a pixel group classified into the blood cell class 81 as the cross section of the lumen 61.

In S103, the control unit 41 of the image processing device 11 displays the three-dimensional data 52 generated in S102 as the three-dimensional image 53 on the display 16. At the time point, the control unit 41 may set an angle at which the three-dimensional image 53 is displayed to any angle.

Specifically, the control unit 41 of the image processing device 11 generates the three-dimensional image 53 based on the three-dimensional data 52 stored in the storage unit 42. The three-dimensional image 53 includes a three-dimensional object group such as a tissue object representing the biological tissue 60 in a three-dimensional space and a medical instrument object representing the medical instrument 62 in the three-dimensional space. That is, the control unit 41 generates a three-dimensional object of the biological tissue 60 based on data of the biological tissue 60 stored in the storage unit 42, and generates a three-dimensional object of the medical instrument 62 based on data of the medical instrument 62 stored in the storage unit 42. The control unit 41 displays the generated three-dimensional image 53 on the display 16 via the output unit 45.

In S104, as a change operation of the user, if there is an operation of setting an angle at which the three-dimensional image 53 is displayed, processing in S105 is executed. If there is no change operation of the user, processing in S106 is executed.

In S105, the control unit 41 of the image processing device 11 receives, via the input unit 44, the operation of setting the angle at which the three-dimensional image 53 is displayed. The control unit 41 adjusts the angle at which the three-dimensional image 53 is displayed to a set angle. In S103, the control unit 41 displays the three-dimensional image 53 on the display 16 at the angle set in S105.

Specifically, the control unit 41 of the image processing device 11 receives, via the input unit 44, an operation of rotating the three-dimensional image 53 displayed on the display 16 by using, for example, the keyboard 14, the mouse 15, or a touchscreen integrally provided with the display 16 by the user. The control unit 41 interactively adjusts the angle at which the three-dimensional image 53 is displayed on the display 16 according to an operation of the user. Alternatively, the control unit 41 receives, via the input unit 44, an operation of inputting a numerical value of the angle at which the three-dimensional image 53 is displayed by using the keyboard 14, the mouse 15, or the touchscreen integrally provided with the display 16 by the user. The control unit 41 adjusts the angle at which the three-dimensional image 53 is displayed on the display 16 according to the input numerical value.

In S106, if there is an update of the tomographic data 51, processing in S107 and S108 is executed. If there is no update of the tomographic data 51, presence or absence of the change operation of the user is confirmed again in S104.

In S107, similar to the processing in S101, the control unit 41 of the image processing device 11 processes the signals input from the probe 20 to newly generate the cross-sectional image 54 of the biological tissue 60, thereby acquiring the tomographic data 51 including at least one new cross-sectional image 54.

In S108, the control unit 41 of the image processing device 11 updates the three-dimensional data 52 of the biological tissue 60 based on the tomographic data 51 acquired in S107. That is, the control unit 41 updates the three-dimensional data 52 based on the tomographic data 51 acquired by the sensor 71. In S108, it is preferable to only update data at a location corresponding to the updated tomographic data 51. In this case, the data processing amount when generating the three-dimensional data 52 can be reduced, and the real-time property of the three-dimensional image 53 can be improved in subsequent S103.

Specifically, the control unit 41 of the image processing device 11 analyzes the cross-sectional image 54 of the biological tissue 60 included in the tomographic data 51 stored in the storage unit 42 for each position in the movement direction of the ultrasound transducer 25 to calculate the centroid position of the cross section of the lumen 61 in the cross-sectional image 54. The control unit 41 executes the smoothing on the calculation results obtained for the plurality of positions in the movement direction of the ultrasound transducer 25. The control unit 41 evaluates the difference between the calculated centroid position and the centroid position obtained as a result of the smoothing for each position in the movement direction of the ultrasound transducer 25, thereby deriving the pulsation score 90 representing the expansion and contraction of the lumen 61 in the cross-sectional image 54 by a numerical value. The control unit 41 determines whether to select the cross-sectional image 54 according to the derived pulsation score 90 for each position in the movement direction of the ultrasound transducer 25. More specifically, when the derived pulsation score 90 is within the setting range, the control unit 41 selects the cross-sectional image 54 as the cross-sectional image 55 used for updating the three-dimensional image 53. On the other hand, when the pulsation score 90 is outside the setting range, the control unit 41 does not select the cross-sectional image 54. The control unit 41 stacks the selected image group obtained for at least some positions among the plurality of positions in the movement direction of the ultrasound transducer 25 and makes the selected image group three-dimensional, thereby updating the three-dimensional data 52 of the biological tissue 60. As the three-dimensional method, a method similar to that of S102 is used. The control unit 41 stores the updated three-dimensional data 52 in the storage unit 42.

Refer to FIG. 7 again for the specific example of the operation of selecting the cross-sectional image 55 when the latest cross-sectional image 54 is acquired. In a case where the cross-sectional image 54 obtained when the ultrasound transducer 25 is at the same position is already selected as the cross-sectional image 55 before time at which the latest cross-sectional image 54 is obtained, and in a case where it is determined in S204 that the pulsation score 90 is within the setting range, the control unit 41 selects the latest cross-sectional image 54 instead of the selected cross-sectional image 54 as the cross-sectional image 55 in S205.

After S108, the control unit 41 of the image processing device 11 displays the three-dimensional data 52 updated in S108 as the three-dimensional image 53 on the display 16 in S103 again.

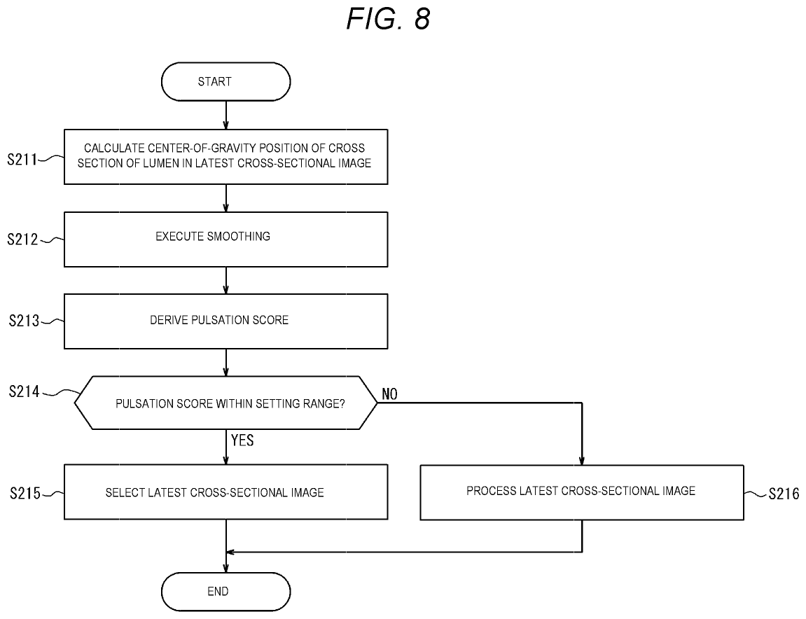

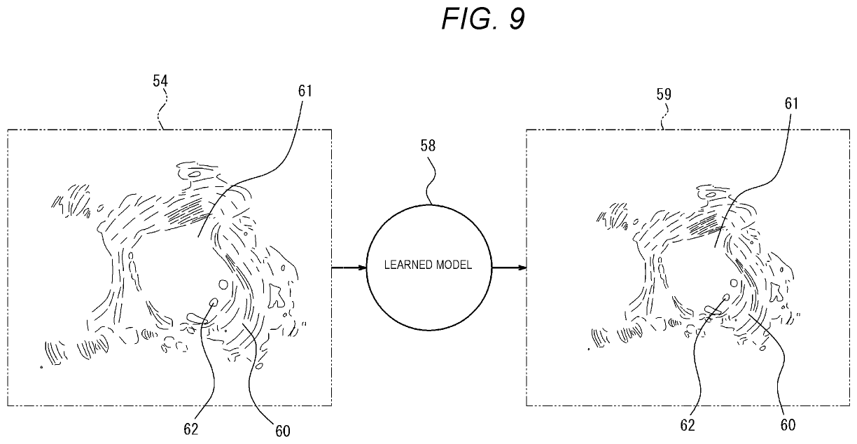

FIG. 8 shows a first modification of the operation of selecting the cross-sectional image 55 when the latest cross-sectional image 54 is acquired. Since processing from S211 to S215 shown in FIG. 8 is similar to the processing from S201 to S205 shown in FIG. 7, description thereof will be omitted. When it is determined in S214 that the pulsation score 90 is outside the setting range, the control unit 41 of the image processing device 11 processes the latest cross-sectional image 54, and selects the obtained processed image as the cross-sectional image 55 in S216. As a method for processing the cross-sectional image 54, a method for processing the cross-sectional image 54 such that the pulsation score 90 approaches the setting range, or preferably a method for processing the cross-sectional image 54 such that the pulsation score 90 falls within the setting range is used. As such a method, for example, as shown in FIG. 9, it is conceivable to process the cross-sectional image 54 by using a learned model 58.

In the example shown in FIG. 9, when the pulsation score 90 calculated in S203 is outside the setting range, the control unit 41 of the image processing device 11 inputs the latest cross-sectional image 54 and the setting range to the learned model 58, and obtains a processed cross-sectional image 59 output from the learned model 58 as a processed image. The processed image is used as the cross-sectional image 55 for generating or updating the three-dimensional image 53. The learned model 58 is trained such that a disguised two-dimensional image can be generated based on a two-dimensional image that is a sample such that the pulsation score 90 approaches the setting range or preferably the pulsation score 90 falls within the setting range by performing the machine learning in advance. The learned model 58 can be generated or updated by, for example, a module that uses artificial intelligence. Examples of such artificial intelligence can include a generative adversarial network.

As described above, for each position in the movement direction of the sensor 71, the control unit 41 of the image processing device 11 may select the acquired cross-sectional image 54 when the derived pulsation score 90 is within the setting range, and may process the cross-sectional image 54 and use the obtained selected image or the processed image for generating or updating the three-dimensional image 53 when the pulsation score 90 is outside the setting range.

According to the modification, even if the selected image group is obtained only for one or some positions among the plurality of positions in the movement direction of the sensor 71, since the processed image group is obtained for remaining positions, it is possible to avoid a situation in which a part of the three-dimensional image 53 may be missing when generating the three-dimensional image 53. It is also possible to avoid a situation in which a part of the three-dimensional image 53 may not be updated and old information may continue to remain when updating the three-dimensional image 53.

The control unit 41 of the image processing device 11 may receive an operation of changing the setting range. In a case where such an operation is received, for each position in the movement direction of the sensor 71, the control unit 41 uses the acquired cross-sectional image 54 for updating the three-dimensional image 53 when the derived pulsation score 90 is within the changed setting range, and uses an image obtained by processing the cross-sectional image 54 for updating the three-dimensional image 53 when the pulsation score 90 is outside the changed setting range.

The control unit 41 of the image processing device 11 may sequentially change the setting range. Every time the setting range is changed, for each position in the movement direction of the sensor 71, the control unit 41 uses the acquired cross-sectional image 54 for updating the three-dimensional image 53 when the derived pulsation score 90 is within the changed setting range, and uses an image obtained by processing the cross-sectional image 54 for updating the three-dimensional image 53 when the pulsation score 90 is outside the changed setting range. For example, by repeating gradually changing the setting range from a relatively small value to a relatively large value, the pulsation can be reproduced on a screen.

The control unit 41 of the image processing device 11 may store a certain number of cross-sectional images 54 in the storage unit 42 for each position in the movement direction of the sensor 71 in preparation for changing the setting range. That is, when the latest cross-sectional image 54 is acquired, the control unit 41 may store the cross-sectional image 54 in the storage unit 42 regardless of whether the cross-sectional image 54 is selected as the cross-sectional image 55 used for generating or updating the three-dimensional image 53. When a certain number of cross-sectional images 54 are already stored in the storage unit 42 for the same position in the movement direction of the sensor 71, the control unit 41 may delete the oldest cross-sectional image 54. When the cross-sectional image 54 having the pulsation score 90 the same as or close to the latest cross-sectional image 54 is already stored in the storage unit 42 for the same position in the movement direction of the sensor 71, the control unit 41 may delete the old cross-sectional image 54 stored in the storage unit 42 for the same position in the movement direction of the sensor 71.

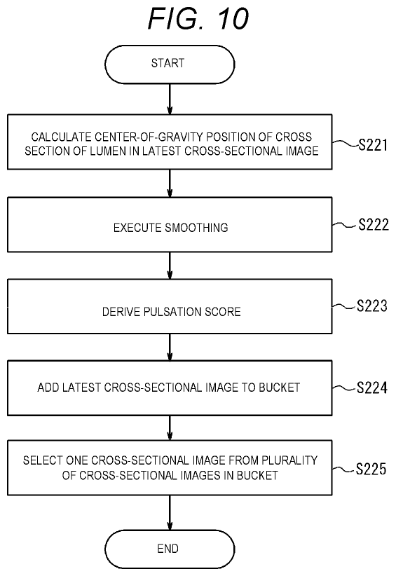

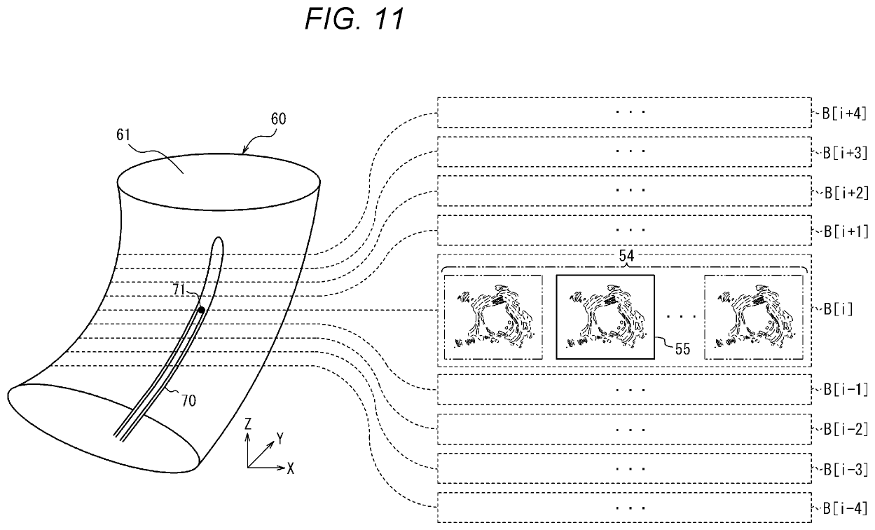

FIG. 10 shows a second modification of the operation of selecting the cross-sectional image 55 when the latest cross-sectional image 54 is acquired. Since processing from S221 to S223 shown in FIG. 10 is similar to the processing from S201 to S203 shown in FIG. 7, description thereof will be omitted. The operation shown in FIG. 10 is not applied to the processing in S102 and is applied only to the processing in S108. In S224, the control unit 41 of the image processing device 11 adds the latest cross-sectional image 54 to a bucket B [i] corresponding to a position in the movement direction of the ultrasound transducer 25 when the latest cross-sectional image 54 is obtained. As shown in FIG. 11, before time at which the latest cross-sectional image 54 is obtained, one or more other cross-sectional images 54 obtained when the ultrasound transducer 25 is at the same position are already stored in the bucket B [i]. Storage regions corresponding to the buckets are set in the storage unit 42 of the image processing device 11. A capacity of each bucket may be optionally set, and can be, for example, four images. If there is no free space in the capacity of the bucket B [i] when an image is added to the bucket B [i], the oldest image may be deleted. When an image is added to the bucket B [i], and in a case where an image having the pulsation score 90 the same as or close to the image is already stored in the bucket B [i], an old image stored in the bucket B may be deleted. When an image is added to the bucket B [i], the pulsation score 90 calculated in S203 may be further stored in association with the image. In each bucket, the cross-sectional images 54 before classification performed by the learned model 56 may be accumulated, or the cross-sectional images 57 after the classification may be accumulated. In S225, the control unit 41 selects, as the cross-sectional image 55, the cross-sectional image 54 whose corresponding pulsation score 90 is within the setting range among the plurality of cross-sectional images 54 in the bucket B [i]. When there is no cross-sectional image 54 whose corresponding pulsation score 90 is within the setting range among the plurality of cross-sectional images 54 in the bucket B [i], nothing may be selected as in the example shown in FIG. 7, or the latest cross-sectional image 54 may be processed as in the example shown in FIG. 8.

As described above, the control unit 41 of the image processing device 11 may acquire the plurality of cross-sectional images 54 that are obtained at time points different from one another by using the sensor 71, and that have different pulsation scores 90 representing the expansion and contraction of the lumen 61 in the cross-sectional images 54 by a numerical value for each position in the movement direction of the sensor 71. The control unit 41 may select the cross-sectional image 54 whose pulsation score 90 is within the setting range among the plurality of acquired cross-sectional images 54 for each position in the movement direction of the sensor 71. The control unit 41 may generate the three-dimensional image 53 based on a selected image group obtained for the plurality of positions in the movement direction of the sensor 71.

According to the modification, the two-dimensional image group used for generating the three-dimensional image 53 is selected according to the pulsation scores 90 representing the expansion and contraction of the lumen 61 in the two-dimensional images by a numerical value, so that it is relatively easy to select the two-dimensional image group acquired at the same timings in the pulsation cycle. As a result, it is possible to reduce the influence of the pulsation when generating the three-dimensional image 53. That is, it is possible to generate an image with almost no unevenness due to the influence of the pulsation as the three-dimensional image 53. Therefore, it is relatively easy for the surgeon to accurately grasp the structure of the biological tissue 60 based on the three-dimensional image 53.

The control unit 41 of the image processing device 11 may receive the operation of changing the setting range. When such an operation is received, for each position in the movement direction of the sensor 71, the control unit 41 uses the cross-sectional image 54 whose pulsation score 90 is within the changed setting range among the plurality of acquired cross-sectional images 54 for updating the three-dimensional image 53.

The control unit 41 of the image processing device 11 may sequentially change the setting range. Every time the setting range is changed, for each position in the movement direction of the sensor 71, the control unit 41 uses the cross-sectional image 54 whose pulsation score 90 is within the changed setting range among the plurality of acquired cross-sectional images 54 for updating the three-dimensional image 53. For example, by repeating gradually changing the setting range from a relatively small value to a relatively large value, the pulsation can be reproduced on the screen.

As a modification of the present embodiment, the control unit 41 of the image processing device 11 may derive the pulsation score 90 by using a dedicated learned model. When a result obtained by measuring an electrocardiogram waveform or an arterial pressure waveform by using an external sensor and the cross-sectional image 54 acquired for each of the plurality of positions in the movement direction of the sensor 71 are input, the dedicated learned model can be trained to output the pulsation score 90 corresponding to the cross-sectional image 54 by performing the machine learning in advance. In the modification, the processing of calculating the centroid position of the cross section of the lumen 61 in the cross-sectional image 54 is unnecessary.

As a modification of the present embodiment, the control unit 41 of the image processing device 11 may derive the pulsation score 90 representing the expansion and contraction of the lumen 61 in the cross-sectional image 54 acquired for each of the plurality of positions in the movement direction of the sensor 71 by a numerical value only based on the result obtained by measuring the electrocardiogram waveform or the arterial pressure waveform by using the external sensor. In the modification, the processing of calculating the centroid position of the cross section of the lumen 61 in the cross-sectional image 54 is unnecessary.

As a modification of the present embodiment, the control unit 41 of the image processing device 11 may acquire the pulsation score 90 from outside instead of deriving the pulsation score 90. As described below, the modification can be applied to the examples shown in FIGS. 7, 8, and 10.

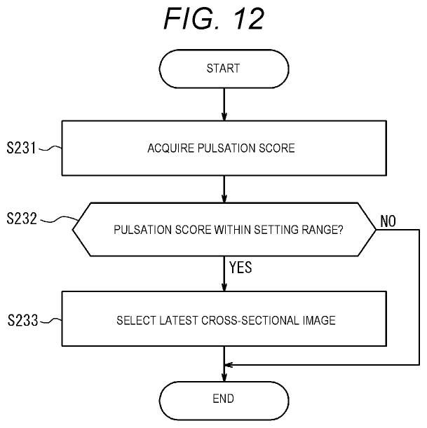

FIG. 12 shows a third modification of the operation of selecting the cross-sectional image 55 when the latest cross-sectional image 54 is acquired. In S231 shown in FIG. 12, the control unit 41 of the image processing device 11 acquires the pulsation score 90 derived by another device from the other device. Since processing in S232 and S233 is similar to the processing in S204 and S205 shown in FIG. 7, description thereof will be omitted.

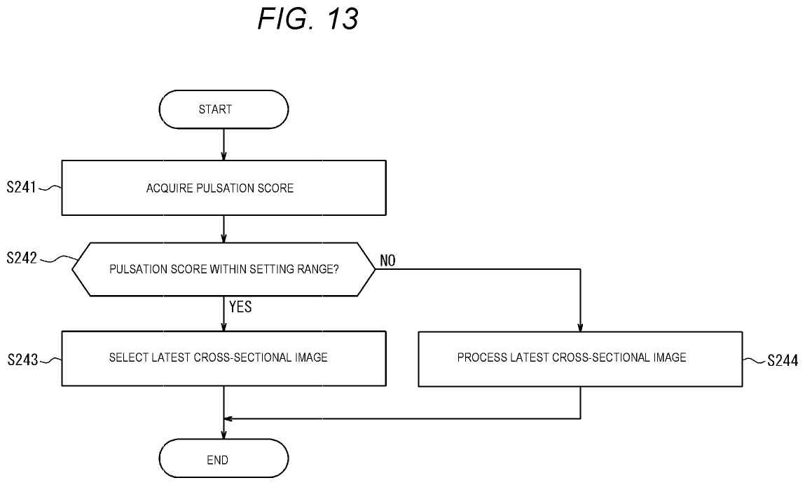

FIG. 13 shows a fourth modification of the operation of selecting the cross-sectional image 55 when the latest cross-sectional image 54 is acquired. Since processing in S241 shown in FIG. 13 is similar to the processing in S231 shown in FIG. 12, description thereof will be omitted. Since processing from S242 to S244 is similar to the processing from S214 to S216 shown in FIG. 8, description thereof will be omitted.

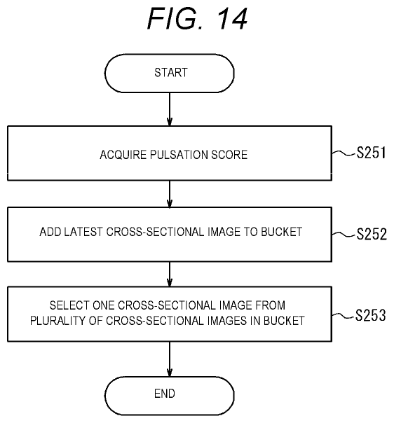

FIG. 14 shows a fifth modification of the operation of selecting the cross-sectional image 55 when the latest cross-sectional image 54 is acquired. Since processing in S251 shown in FIG. 14 is similar to the processing in S231 shown in FIG. 12, description thereof will be omitted. Since processing in S252 and S253 is similar to the processing in S224 and S225 shown in FIG. 10, description thereof will be omitted.

The present disclosure is not limited to the embodiment described above. For example, two or more blocks disclosed in a block diagram may be combined, or one block may be divided. Instead of executing the two or more steps or processes disclosed in a flowchart in a chronological order according to the description, the steps or processes may be executed in parallel or in a different order depending on a processing capability of a device that executes the, or when necessary. In addition, modifications can be made in a scope not departing from the gist of the present disclosure.

The detailed description above describes embodiments of an image display device, an image display system, an image processing method, and an image processing program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An image processing device comprising:
a control unit configured to:
acquire a cross-sectional image obtained using a sensor configured to move in a lumen of a biological tissue for each position in a movement direction of the sensor;
analyze the acquired cross-sectional image to calculate a centroid position of a cross section of the lumen in the cross-sectional image;
execute smoothing on a calculation result obtained for a plurality of positions in the movement direction of the sensor;
evaluate a difference between the calculated centroid position and a centroid position obtained as a result of the smoothing for each position in the movement direction of the sensor; and
derive a pulsation score representing expansion and contraction of the lumen in the acquired cross-sectional image by a numerical value.

2. The image processing device according to claim 1, wherein the control unit is configured to determine whether to select an acquired cross-sectional image according to a derived pulsation score for each position in the movement direction of the sensor, and generate a three-dimensional image representing the biological tissue based on a selected image group obtained for at least some positions among the plurality of positions.

3. The image processing device according to claim 2, wherein for each position in the movement direction of the sensor, the control unit is configured to select an acquired cross-sectional image, and uses the obtained selected image for generating or updating the three-dimensional image when a derived pulsation score is within a setting range, and does not select the cross-sectional image when the pulsation score is outside the setting range.

4. The image processing device according to claim 2, wherein for each position in the movement direction of the sensor, the control unit is configured to:
select an acquired cross-sectional image, and use the obtained selected image for generating or updating the three-dimensional image when a derived pulsation score is within a setting range; and
process the cross-sectional image and use the obtained processed image for generating or updating the three-dimensional image when the pulsation score is outside the setting range.

5. The image processing device according to claim 4, wherein when an operation of changing the setting range is received, for each position in the movement direction of the sensor, the control unit is configured to:
use an acquired cross-sectional image for updating the three-dimensional image when a derived pulsation score is within a changed setting range; and
use an image obtained by processing the cross-sectional image for updating the three-dimensional image when the pulsation score is outside the changed setting range.

6. The image processing device according to claim 4, wherein the control unit is configured to sequentially change the setting range, and every time the setting range is changed, for each position in the movement direction of the sensor, the control unit is configured to use an acquired cross-sectional image for updating the three-dimensional image when a derived pulsation score is within a changed setting range, and use an image obtained by processing the cross-sectional image for updating the three-dimensional image when the pulsation score is outside the changed setting range.

7. The image processing device according to claim 4, wherein for each position in the movement direction of the sensor, the control unit is configured to input an acquired cross-sectional image and the setting range to a learned model, and obtain a processed cross-sectional image output from the learned model as the processed image when a derived pulsation score is outside the setting range.

8. The image processing device according to claim 7, wherein the control unit is configured to use, as the learned model, a model generated or updated by a module that uses artificial intelligence.

9. An image display system comprising:
the image processing device according to claim 2; and
a display configured to display the three-dimensional image.

10. An image processing device comprising:
a control unit configured to:
acquire a cross-sectional image obtained using a sensor configured to move in a lumen of a biological tissue and a pulsation score representing expansion and contraction of the lumen in the cross-sectional image by a numerical value for each position in a movement direction of the sensor;
select the cross-sectional image when the acquired pulsation score is within a setting range;
process the cross-sectional image when the pulsation score is outside the setting range; and
generate a three-dimensional image representing the biological tissue based on a selected image group and a processed image group obtained for a plurality of positions in the movement direction of the sensor.

11. The image processing device according to claim 10, wherein when an operation of changing the setting range is received, for each position in the movement direction of the sensor, the control unit is configured to:
use an acquired cross-sectional image for updating the three-dimensional image when an acquired pulsation score is within a changed setting range; and
use an image obtained by processing the cross-sectional image for updating the three-dimensional image when the pulsation score is outside the changed setting range.

12. The image processing device according to claim 10, wherein the control unit is configured to sequentially change the setting range, and every time the setting range is changed, for each position in the movement direction of the sensor, the control unit is configured to use an acquired cross-sectional image for updating the three-dimensional image when an acquired pulsation score is within a changed setting range, and use an image obtained by processing the cross-sectional image for updating the three-dimensional image when the pulsation score is outside the changed setting range.

13. The image processing device according to claim 10, wherein for each position in the movement direction of the sensor, the control unit is configured to input an acquired cross-sectional image and the setting range to a learned model, and obtain a processed cross-sectional image output from the learned model as a processed image when an acquired pulsation score is outside the setting range.

14. The image processing device according to claim 13, wherein the control unit is configured to use, as the learned model, a model generated or updated by a module that uses artificial intelligence.

15. An image display system comprising:
the image processing device according to claim 10; and
a display configured to display the three-dimensional image.

16. An image processing device comprising:
a control unit configured to:
acquire a plurality of cross-sectional images that are obtained at time points different from one another by using a sensor configured to move in a lumen of a biological tissue and that have different pulsation scores representing expansion and contraction of the lumen in the cross-sectional images by a numerical value for each position in a movement direction of the sensor;
select a cross-sectional image whose pulsation score is within a setting range among the plurality of acquired cross-sectional images; and
generate a three-dimensional image representing the biological tissue based on a selected image group obtained for a plurality of positions in the movement direction of the sensor.

17. The image processing device according to claim 16, wherein when an operation of changing the setting range is received, for each position in the movement direction of the sensor, the control unit is configured to use a cross-sectional image whose pulsation score is within a changed setting range among the plurality of acquired cross-sectional images for updating the three-dimensional image.

18. The image processing device according to claim 16, wherein the control unit is configured to sequentially change the setting range, and every time the setting range is changed, for each position in the movement direction of the sensor, the control unit is configured to use a cross-sectional image whose pulsation score is within a changed setting range among the plurality of acquired cross-sectional images for updating the three-dimensional image.

19. The image processing device according to claim 16, wherein for each position in the movement direction of the sensor, the control unit is configured to input an acquired cross-sectional image and the setting range to a learned model, and obtain a processed cross-sectional image output from the learned model as a processed image when an acquired pulsation score is outside the setting range.

20. The image processing device according to claim 19, wherein the control unit is configured to use, as the learned model, a model generated or updated by a module that uses artificial intelligence.
